(19) United States
(12) Patent Application Publication
(10) Pub. No.: US 2009/0318453 A1
(43) Pub. Date: Dec. 24, 2009

(54) POSTOPERATIVE ADJUVANT CHEMOTHERAPY FOR GASTRIC CANCER

(75) Inventor: Koichi Okabe, Chiyoda-ku (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: TAIHO PHARMACEUTICAL CO., LTD, Chiyoda-ku (JP)

(21) Appl. No.: 12/442,308

(22) PCT Filed: Sep. 20, 2007
(86) PCT No.: PCT/JP2007/001018

§ 371 Date: Mar. 20, 2009

(30) Foreign Application Priority Data
Sep. 22, 2006 (JP) 2006-257835
Jan. 18, 2007 (JP) 2007-009358

(51) Int. Cl. A61K 31/53

(52) U.S. Cl. 514/241

(57) ABSTRACT

The invention provides a method for treatment of gastric cancer, which method is a postoperative adjuvant chemotherapy for gastric cancer, comprising orally administering, to a patient with gastric cancer in stage II, IIIA, or IIIB in a classification of the stage of gastric cancer, a pharmaceutical composition containing Tegafur, Gimeracil, and Oteracil potassium at a molar ratio of 1:0.4:1, at a dose of 50 to 150 mg/day as Tegafur dose and according to an administration schedule including, from within 45 days after a surgical operation of gastric cancer, drug administration for 28 consecutive days, followed by a rest period of 7 to 14 consecutive days.

The chemotherapy reduces the relapse rate after a surgical operation, thereby improving the survival rate.

### TECHNICAL FIELD

The present invention relates to a postoperative adjuvant chemotherapy for gastric cancer.

### BACKGROUND ART

In Japan, gastric cancer is the cancer having the highest morbidity. Although the number of deaths by gastric cancer has gradually decreased in recent years, gastric cancer still accounts for 20% of all deaths by cancer. Gastric cancer is mainly treated through a surgical operation, and the cancer at an early stage (stage I) can be virtually completely cured through surgery alone, with a five-year survival rate of 90% or higher. However, in the case where the cancer has progressed moderately (e.g., stage II (excluding T1) or III), recurrence unavoidably follows, even when curative resection (curability A or B) was performed. Thus, in order to enhance cure rate, effective therapeutic methods for preventing postoperative recurrence must be developed. Among such therapeutic methods, postoperative adjuvant chemotherapy is a promising candidate for gastric cancer, since the therapy chemically or systemically attacks small metastatic foci remaining after surgery, and its effect on prognosis has already been established for breast cancer and colon cancer.

For the development of postoperative adjuvant chemotherapy for gastric cancer, there have been carried out a variety of controlled clinical trials employing combination chemotherapy effective for advanced or recurrent cancer with respect to a surgery-only group as a control. However, the efficacy thereof has not yet been established. In 1993, Hermans et al. carried out meta-analysis, in terms of postoperative adjuvant chemotherapy, for the results of 11 tests (2,096 cases) carried out mainly in Western countries. In the meta-analysis, no statistical significance was obtained (odds ratio: 0.88 [95% CI: 0.72-1.08]); i.e., no standard postoperative adjuvant chemotherapy was found, and further trials with respect to a surgery group were found to be essential (Non-Patent Document 1). Thereafter, new reports (2 tests, 318 cases) from Japan were added to the results of the above tests, and meta-analysis was performed again. However, through evaluation, the new results (odds ratio: 0.82 [95% CI: 0.68-0.98]) support the efficacy of postoperative adjuvant chemotherapy, but are not employed as sufficient evidence for the chemotherapy to serve as a standard therapeutic method (Non-Patent Document 2). Further, the meta-analytical results of 13 tests (1,990 cases) (excluding the results obtained in Asian regions) were carried out by Earle et al. (odds ratio: 0.80 [95% CI: 0.66-0.97]) (Non-Patent Document 3), and academic reports by Floriani in 2000 including the results of 20 tests (3,510 cases) (odds ratio: 0.83 [95% CI: 0.76-0.90]) were the same as mentioned above (Non-Patent Document 4). In postoperative adjuvant chemotherapy trials carried out in Western countries, the five-year survival rate of surgery-only groups (control) was as low as 20 to 40%, and the efficacy of postoperative adjuvant chemotherapy was not proven in combination chemotherapy, which is effective for advanced or recurrent gastric cancer. A possible reason for the low five-year survival rate is as follows. In Western countries, systematic lymph node dissection is not generally performed, and a considerable amount of tumor residues remain, which makes the postoperative adjuvant chemotherapy impotent. Although there was an additional report from European countries including a study on the efficacy of various combination therapies, the reported trial was performed on a small scale and targeted only to lymph node metastasis positive cases. Therefore, at present, new clinical trials of postoperative adjuvant chemotherapy employing a new drug, along with preoperative drug administration and radiotherapy, with respect to a surgery-only group, are under investigation (Non-Patent Documents 5 to 7).

In Japan, Nakajima et al. previously carried out meta-analysis of 1,117 gastric cancer cases, which had been enrolled as subjects for six trials (10 administration methods) held in Cancer Institute Hospital, Japanese Foundation for Cancer Research, and reported that a therapeutic method mainly including systemic administration of Mitomycin C (MMC) and a 5-FU drug after the relevant curative resection prolongs the survival duration (odds ratio: 0.63 [95% CI: 0.51-0.79]) (Non-Patent Document 8). In 1999, the Japan Clinical Oncology Group (JCOG) reported the five-year survival rate of serosa-negative gastric cancer cases, determined through the JCOG 8801 study, in which the efficacy of MMC+5-FU+UFT on postoperative adjuvant chemotherapy was evaluated as compared with a surgery-only group as a control. According to the report, the five-year survival rate of the postoperative adjuvant chemotherapy group was 85.8%, and that of the surgery-only group was 82.9%. Therefore, the efficacy on postoperative adjuvant chemotherapy was not proven (Non-Patent Document 9). JCOG further carried out the JCOG 9206 study for the similar subjects. However, in this test, the efficacy of MFC (MMC+5-FU+AraC)+5-FU (oral) over the surgery-only group was not proven (postoperative adjuvant chemotherapy: 91.2%, surgery-only: 86.1%, p=0.13) (Non-Patent Document 10).

Meanwhile, a composition containing Tegafur (1-(2-tetrahydrofuryl)-5-fluorouracil) and uracil, which potentiates the antitumor effect of Tegafur, at a mole ratio of 1:4, is a commercial product called “UFT” and is used as a cancer chemotherapy agent. This composition was previously used in postoperative adjuvant chemotherapy for gastric cancer by administering for consecutive days at a dose of 375 mg/m²/day as Tegafur dose. However, administration of the composition without any other drugs failed to demonstrate a significant survival benefit (Non-Patent Document 11). When MMC and the above composition were administered every day at a dose of 600 mg/day as Tegafur dose for two years from the day two weeks after surgery, a significant survival benefit was proven. In this case, however, adverse events (leukopenia: 22%, elevated GOT: 18%, elevated GPT: 18%, anorexia: 25%, nausea and vomiting: 22%, and diarrhea: 7%) were observed. In the drug administration, the median of the administration period remains as low as 16.9% (123 days) of the target administration period. In addition, the sole administration of the composition has never been reported to demonstrate a significant survival benefit (Non-Patent Document 12).

Another composition containing Tegafur, Gimeracil, which potentiates the antitumor effect of Tegafur, and Oteracil potassium, which reduce the gastrointestinal toxic, at a molar ratio of 1:0.4:1 is a commercial product called “TS-1” and is used as a cancer chemotherapy agent. Actually, applicability of TS-1 to postoperative adjuvant chemotherapy for gastric cancer has been investigated. The investigation has found that one-year administration of TS-1 after surgery is considered to be applicable to postoperative adjuvant chemotherapy, although slightly higher side effect expression caused by gastrectomy was observed as compared with the treatment of non-resectable or recurrent gastric cancer. However, the survival benefit of TS-1 has never been reported, including survival rate (Non-Patent Document 13).
Non-Patent Document 1: Hermans, J. et al., J. Clin. Oncol. 11(8): 1441-1447, 1993
Non-Patent Document 2: Hermans, J., J. Clin. Oncol. 12(4): 879-880, 1994
Non-Patent Document 3: Earle, C. C. et al., Eur. J. Cancer 35(7): 1059-1064, 1999
Non-Patent Document 4: Floriani, I. et al., Proc. ASCO 19: 262a, 2000
Non-Patent Document 5: Cirera, L. et al., J. Clin. Oncol. 17(12): 3810-3815, 1999
Non-Patent Document 6: Neri, B. et al., Brit. J. Cancer 84(7): 878-880, 2001
Non-Patent Document 7: Valle, J. W., Brit. J. Cancer 84(7): 875-877, 2001
Non-Patent Document 8: Toshifusa Nakajima et al., Japanese Journal of Cancer and Chemotherapy, 21(11): 1800-1805, 1994
Non-Patent Document 9: Nakajima, T., et al., Lancet 354: 273-277, 1999
Non-Patent Document 10: Nashimoto, A. et al., Proc. ASCO 20: 164a, 2001
Non-Patent Document 11: Tokunaga, T. et al., J. Surg. Oncol. 75: 31-36, 2000
Non-Patent Document 12: Arima, S. et al., Eur. J. Surg. 160: 227-232, 1994
Non-Patent Document 13: Kinoshita, T. et al., Gastric Cancer 7: 104-109, 2004

### DISCLOSURE OF THE INVENTION

### Problems to be Solved by the Invention

An object of the present invention is to provide a postoperative adjuvant chemotherapy for a patient with gastric cancer in stage II, IIIA, or IIIB in a classification of the stage of gastric cancer, which chemotherapy reduces the relapse rate after a surgical operation, thereby improving the survival rate.

### Means for Solving the Problems

In view of the foregoing, the present inventor has carried out extensive studies on postoperative adjuvant chemotherapy for gastric cancer in terms of target patients, the dose thereof, the administration schedule, etc., and has found that through orally administering, to a patient with gastric cancer in stage II, IIIA, or IIIB in a classification of the stage of gastric cancer, a pharmaceutical composition containing Tegafur, Gimeracil, and Oteracil potassium at a molar ratio of 1:0.4:1, at a dose of 50 to 150 mg/day as Tegafur dose and according to an administration schedule including, from within 45 days after a surgical operation of gastric cancer, drug administration for 28 consecutive days, followed by a rest period of 7 to 14 consecutive days, the pharmaceutical composition can be administered to the patient for a long period of time reasonably, incidence of adverse effects is reduced, and the survival rate of the patient is significantly increased. The present invention has been accomplished on the basis of this finding.

Accordingly, the present invention provides a method for treatment of gastric cancer, which method is a postoperative adjuvant chemotherapy for gastric cancer, comprising orally administering, to a patient with gastric cancer in stage II, IIIA, or IIIB in a classification of the stage of gastric cancer, a pharmaceutical composition containing Tegafur, Gimeracil, and Oteracil potassium at a molar ratio of 1:0.4:1, at a dose of 50 to 150 mg/day as Tegafur dose and according to an administration schedule including, from within 45 days after a surgical operation of gastric cancer, drug administration for 28 consecutive days, followed by a rest period of 7 to 14 consecutive days.

The present invention also provides a pharmaceutical composition for treatment of gastric cancer, comprising Tegafur, Gimeracil, and Oteracil potassium at a molar ratio of 1:0.4:1, wherein the composition is for oral administration, to a patient with gastric cancer in stage II, IIIA, or IIIB in a classification of the stage of gastric cancer, at a dose of 50 to 150 mg/day as Tegafur dose and according to an administration schedule including, from within 45 days after a surgical operation of gastric cancer, drug administration for 28 consecutive days, followed by a rest period of 7 to 14 consecutive days.

The present invention also provides use of a composition comprising Tegafur, Gimeracil, and Oteracil potassium at a molar ratio of 1:0.4:1, for producing a therapeutic drug for gastric cancer, wherein the therapeutic drug for gastric cancer is for oral administration to a patient with gastric cancer in stage II, IIIA, or IIIB in a classification of the stage of gastric cancer, at a dose of 50 to 150 mg/day as Tegafur dose and according to an administration schedule including, from within 45 days after a surgical operation of gastric cancer, drug administration for 28 consecutive days, followed by a rest period of 7 to 14 consecutive days.

### EFFECTS OF THE INVENTION

According to the therapy of the present invention, the survival rate of a gastric cancer patient after a surgical operation is significantly improved, and the cancer relapse rate is significantly reduced. Also, according to the dose and administration schedule employed in the therapy of the present invention, the pharmaceutical composition can be administered to a patient for a long period of time reasonably, and incidence of adverse effects is reduced.

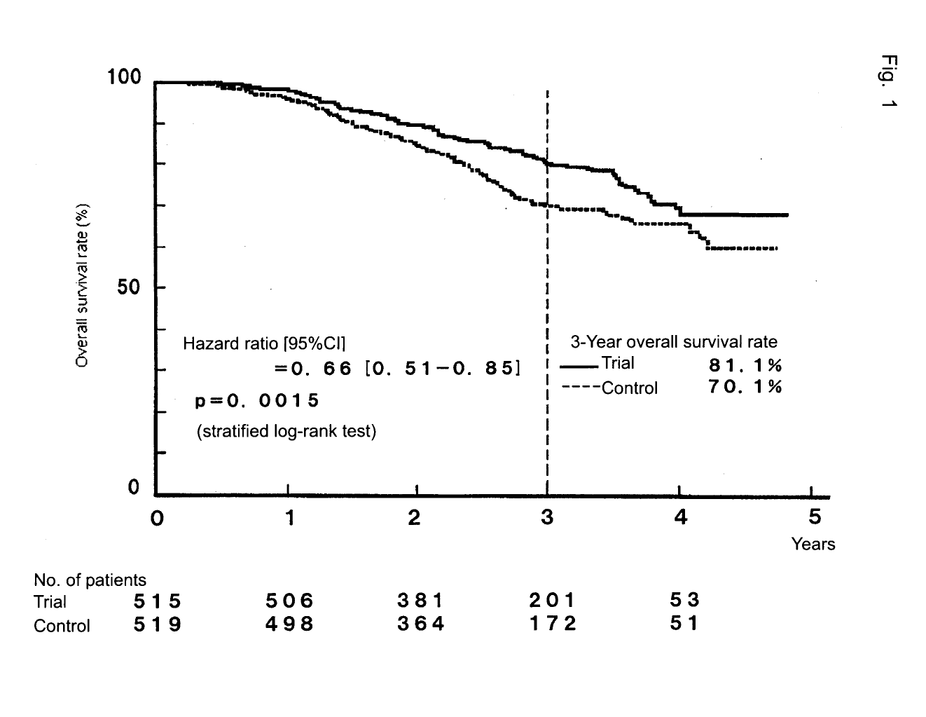

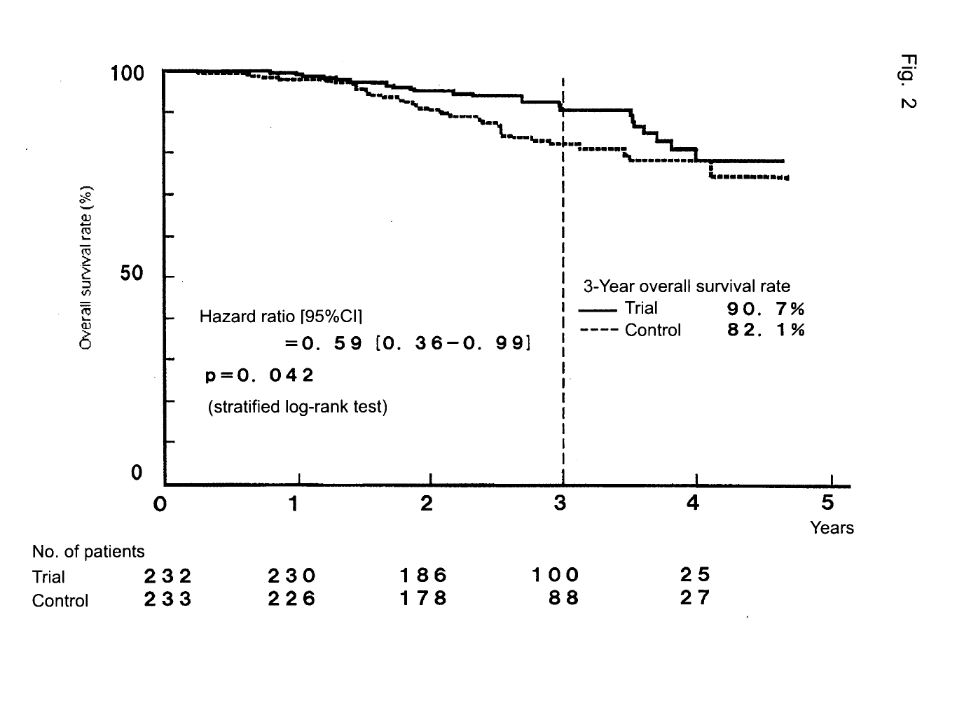

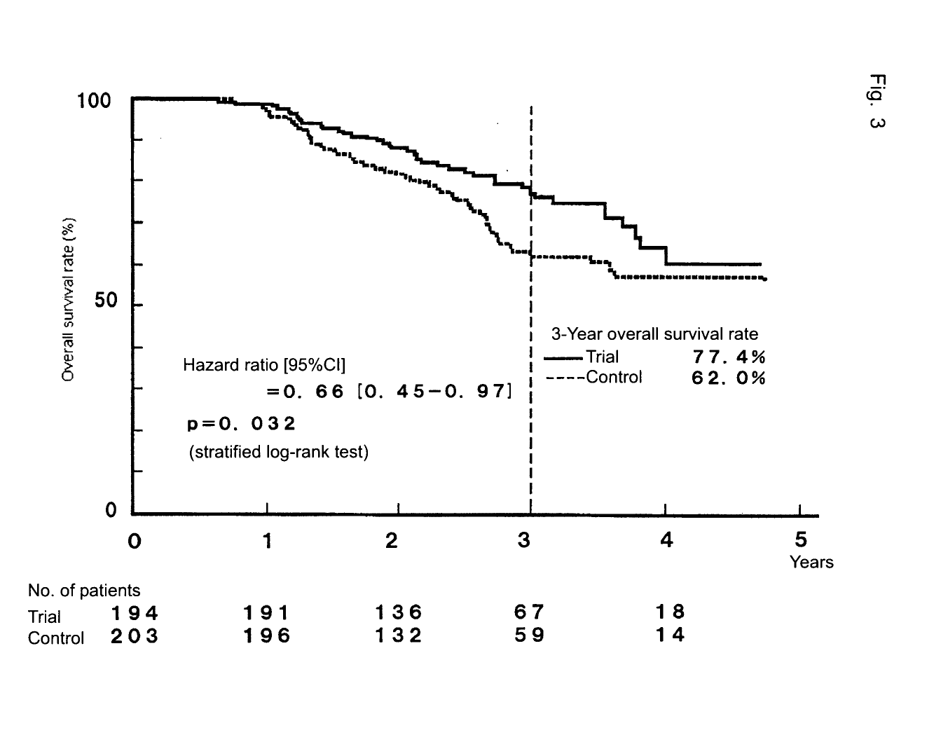

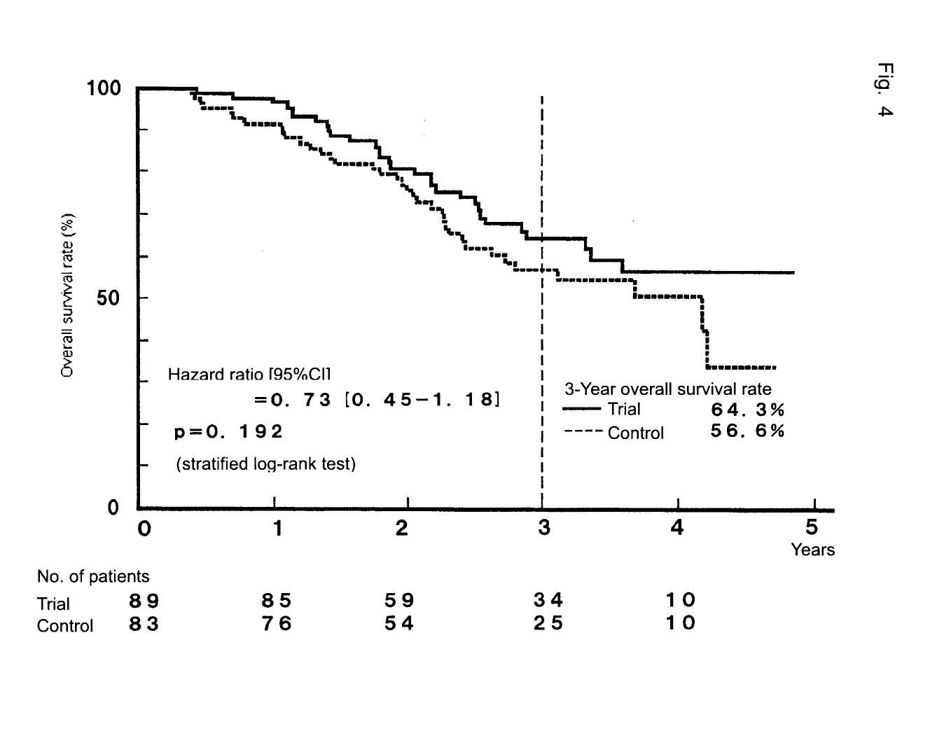

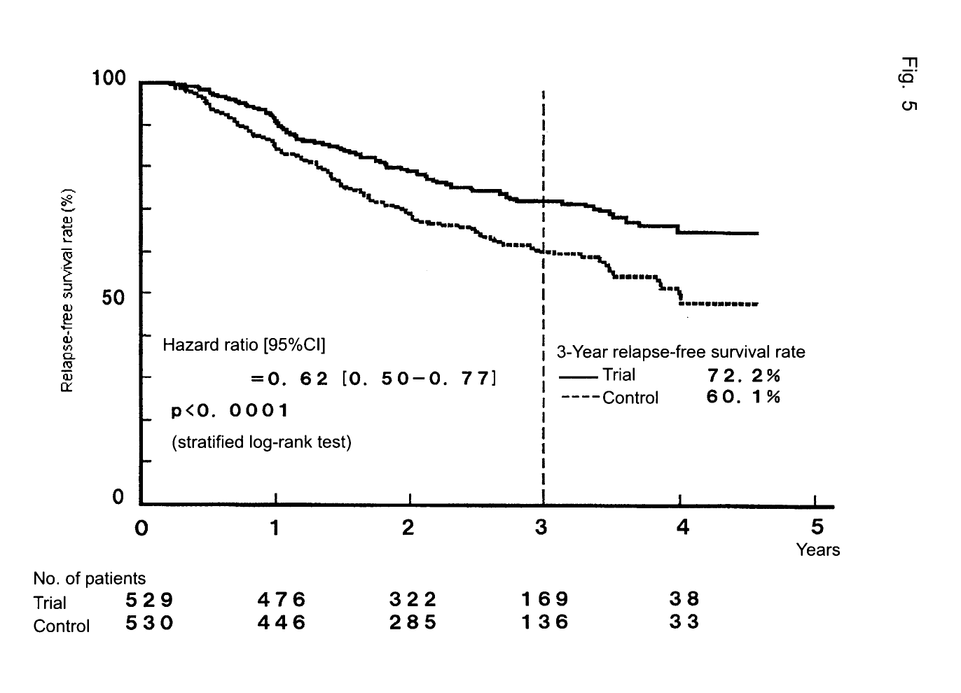

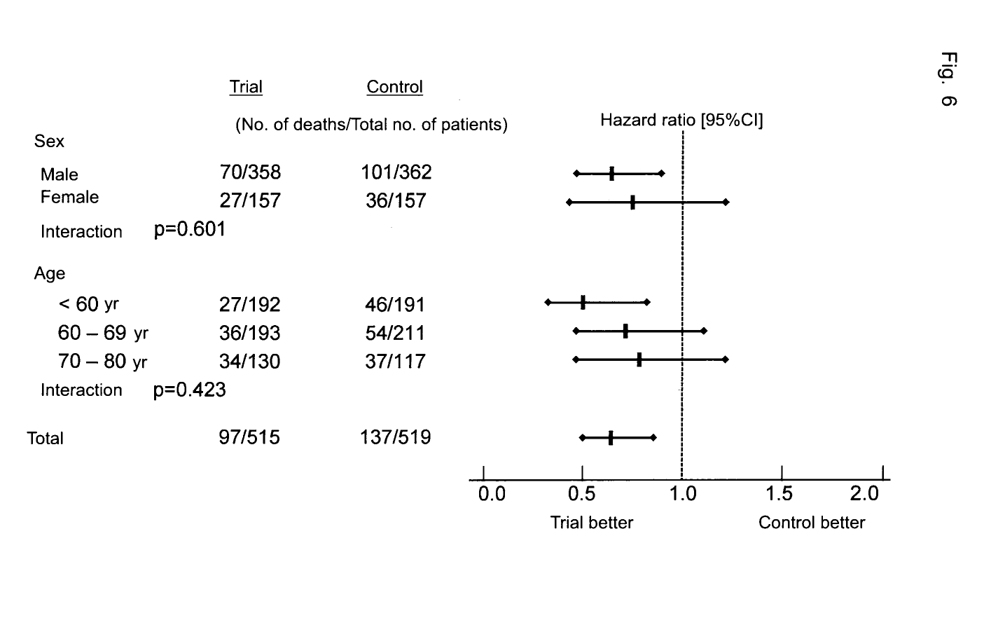

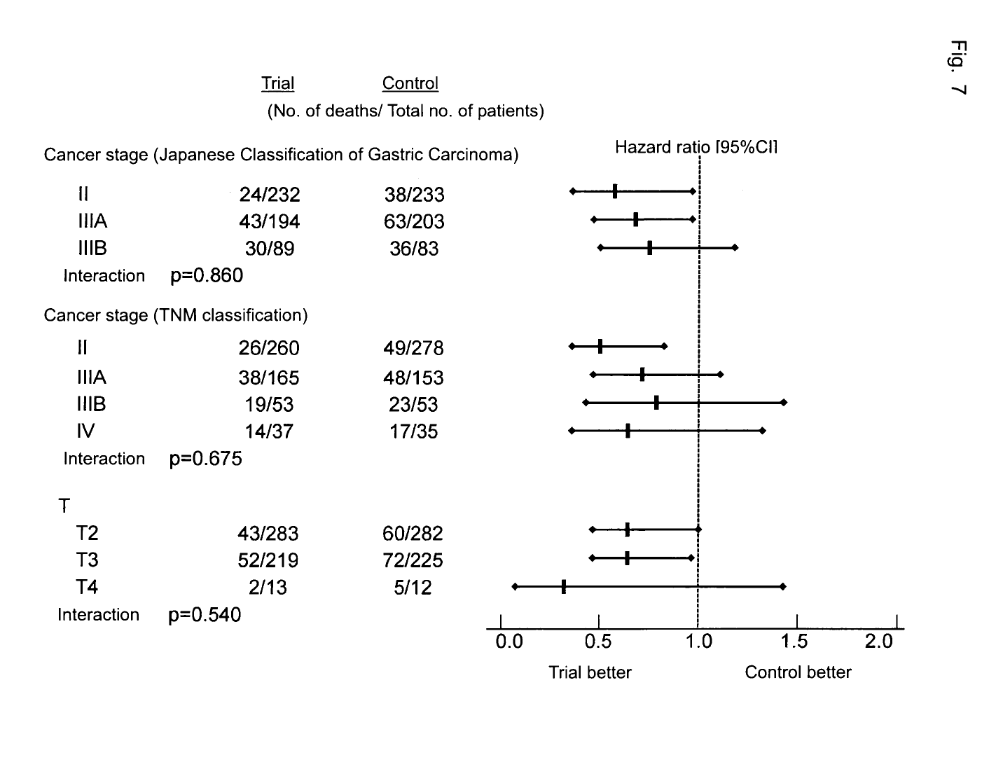

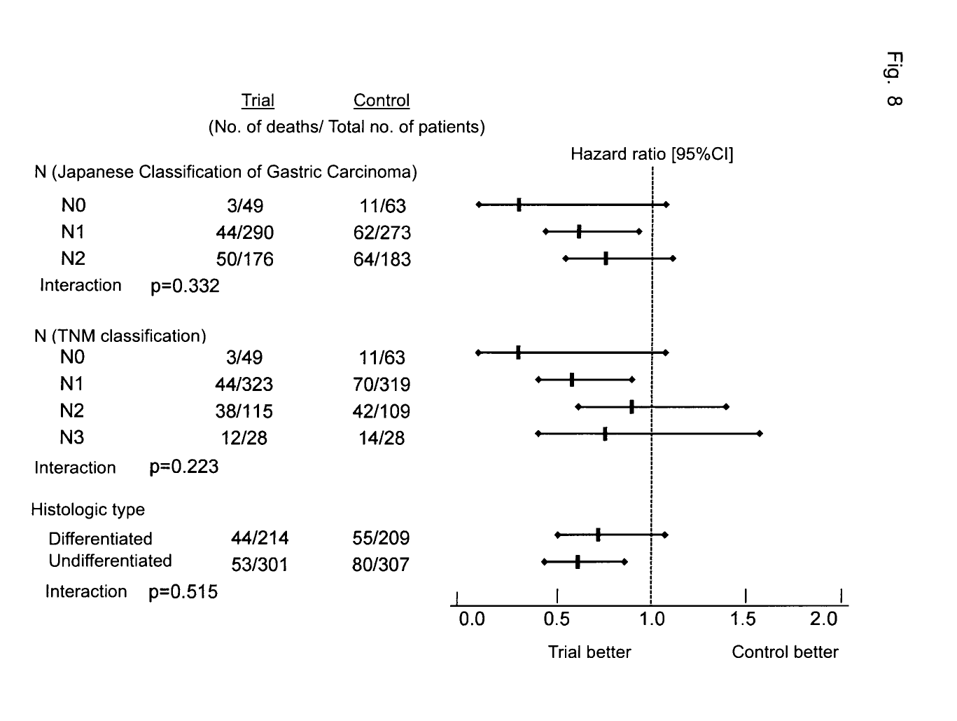

### BRIEF DESCRIPTION OF THE DRAWINGS

[FIG. 1] A graph showing the change in overall survival rate of all cases that had received the therapy of the present invention.

[FIG. 2] A graph showing the change in overall survival rate of the stage II patients which had received the therapy of the present invention.

[FIG. 3] A graph showing the change in overall survival rate of the stage IIIA patients which had received the therapy of the present invention.

[FIG. 4] A graph showing the change in overall survival rate of the stage IIIB patients which had received the therapy of the present invention.

[FIG. 5] A graph showing the change in relapse-free survival rate of all cases that had received the therapy of the present invention.

[FIG. 6] A graph and data showing the analytical results of the therapy of the present invention in terms of sex and age of the patients.

[FIG. 7] A graph and data showing the analytical results of the therapy of the present invention in terms of the TNM classification and depth of tumor invasion (T).

[FIG. 8] A graph and data showing the analytical results of the therapy of the present invention in terms of lymph node metastasis (N) and histologic type.

### BEST MODES FOR CARRYING OUT THE INVENTION

In the present invention, the gastric cancer patients to which the therapy of the invention is applied are patients with gastric cancer in stage II, IIIA, or IIIB in a classification of the stage of gastric cancer. These patients generally receive typical surgery; i.e., resection of ≧⅔ portions of the stomach and D2 lymph node dissection. The tissues removed through the surgery may be analyzed through histological observation. The aforementioned classification of the extent of cancer is based on the classification by Japanese Gastric Cancer Association; i.e., Japanese Classification of Gastric Carcinoma, 13th edition, edited by Japanese Gastric Cancer Association, Kanehara & Co., Ltd. (Tokyo), 1999.

The chemotherapeutic agent used in the present invention is a pharmaceutical composition comprising Tegafur, Gimeracil, and Oteracil potassium at a molar ratio of 1:0.4:1 (Japanese Patent No. 2614164). The pharmaceutical composition, which is a commercial product “TS-1,” is used as a therapeutic drug for gastric cancer, colorectal cancer, head and neck cancer, non-small cell lung cancer, non-operable or recurrent breast cancer, and pancreatic cancer. However, as described in a package insert of TS-1, efficacy of TS-1 on the postoperative adjuvant chemotherapy for gastric cancer patients has not been established.

No particular limitation is imposed on the form of the composition, so long as the composition for oral administration contains Tegafur, Gimeracil, and Oteracil potassium. Examples of the form include tablet, coated tablet, granules, fine granules, powder, capsule, pill, emulsion, suspension, and liquid.

In preparation of tablets from the composition, an excipient, a binder, a disintegrant, a disintegration inhibitor, an absorption enhancer, a humectant, an adsorbent, a lubricant, and the like may be used. Examples of the excipient include lactose, saccharose, sodium chloride, glucose, urea, starch, calcium carbonate, kaolin, crystalline cellulose, and silicic acid. Examples of the binder include water, ethanol, propanol, simple syrup, liquid glucose, liquid starch, gelatin solution, carboxymethylcellulose, shellac, methylcellulose, potassium phosphate, and polyvinylpyrrolidone. Examples of the disintegrant include dry starch, sodium alginate, powdered agar, powdered laminaran, sodium hydrogencarbonate, calcium carbonate, polyoxyethylene sorbitan fatty acid esters, sodium lauryl sulfate, stearic acid monoglyceride, starch, and lactose. Examples of the disintegration inhibitor include saccharose, stearic acid, cacao butter, and hydrogenated oil. Examples of the absorption enhancer include quaternary ammonium salts and sodium lauryl sulfate. Examples of the humectant include glycerin and starch. Examples of the adsorbent include starch, lactose, kaolin, bentonite, and colloidal silicic acid. Examples of the lubricant include purified talc, stearic acid salts, powdered boric acid, and polyethylene glycol. Furthermore, if necessary, the tablets may be modified to form coated tablets having a standard coating; e.g., sugar-coated tablets, gelatin-coated tablets, enteric-coated tablets, film-coated tablets, double-layer tablets, and multi-layer tablets.

In preparation of pills, there may be used, for example, excipients such as glucose, lactose, starch, cacao butter, hydrogenated vegetable oil, kaolin, and talc; binders such as powdered gum arabic, powdered tragacanth, gelatin, and ethanol; and disintegrants such as powdered laminaran and powdered agar.

Capsule products may be prepared through mixing Tegafur, Gimeracil, and Oteracil potassium, with the aforementioned various carriers, and filling hard gelatin capsules or hard capsules with the mixture.

Into the aforementioned pharmaceutical products, if necessary, a coloring agent, a preservative, a perfume, a flavoring agent, a sweeting agent, and the like and other drugs may be incorporated.

So long as the pharmaceutical composition contains Tegafur, Gimeracil, and Oteracil potassium at a molar ratio of 1:0.4:1, one pharmaceutical product produced therefrom may contain these three ingredients in the product. Alternatively, these three ingredients may be formed into different forms of pharmaceutical products (e.g., tablets and capsules).

The composition of the invention is administered orally. The dose of the composition, is 50 to 150 mg/day as Tegafur dose, particularly preferably 80 to 120 mg/day. When the dose falls outside the range of 50 to 150 mg/day, in some cases, efficacy of the composition cannot be fully attained, or adverse effects increase. Notably, the dose is a daily dose, and a daily dose of 50 to 150 mg as Tegafur dose is preferably divided into two doses. For example, the composition is preferably administered after breakfast and evening meal.

The administration is started within 45 days after the relevant surgical operation. When the administration is started after day 45, the efficacy of the therapy of the present invention cannot be fully attained. Therefore, the administration is preferably started within 45 days after the surgical operation and at the timing when the composition can be orally administered to the patient.

In the present invention, it is important that the administration schedule includes 28 consecutive days of drug administration, followed by a rest period of 7 to 14 consecutive days. In order to fully attain the therapeutic effect, the composition is preferably continuously administered to the patient every day. However, such continuous administration imposes a burden on the patient, and adverse effects may increase. In contrast, through an administration schedule including drug administration for 28 consecutive days, followed by a rest period of 7 to 14 consecutive days, and repetition of the set of administration and rest, the composition can be administered to the patient for a long period of time without imposing a heavy burden. When the administration period is in excess of 28 consecutive days, the burden on the patient increases, whereas when the administration period is shorter than 28 consecutive days, a sufficient therapeutic effect may fail to be attained. Provision of a rest period of 7 to 14 consecutive days results in reduction of adverse effects. The rest period is preferably 14 days. Unless safety is impaired, the rest period may be shortened. Even in such a case, at least 7 days are provided as a rest period. When the rest period is shorter than 7 days, adverse effects cannot be sufficiently cured, and in some cases, subsequent continuous drug administration cannot be performed. By virtue of decrease of burden on the patient and reduction of adverse effects, long-term administration of the composition can be realized.

The patient who receives the therapy of the invention is preferably a patient with gastric cancer in stage II or IIIA in a classification of the stage of gastric cancer.

The administration period is preferably one-year after the relevant surgical operation. Through such one year administration, a satisfactory therapeutic effect can be attained.

Through the postoperative adjuvant chemotherapy for gastric cancer according to the present invention, the postoperative relapse rate is considerably lowered, and long-survival rate significantly improves.

### EXAMPLES

Examples are given below to describe the invention in more detail, but the scope of the invention is not limited by the Examples.

### Example 1

### (1) Purposes of the Trial

TS-1 (capsule form) is administered to gastric cancer patients in stage II (excluding T1 cases), IIIA, or IIIB who received curative resection, and the survival benefit of the drug is assessed and compared with that of a surgery-only group serving as a control group, whereby the efficacy of the postoperative adjuvant chemotherapy is assessed. The evaluation is performed on the basis of overall survival as a primary end point and relapse-free survival and safety of TS-1 administration as secondary end points.

### (2) Target Cases

### (i) Eligibility Criteria

1) A case in which gastric cancer was histologically proven.
2) A case which received ≧D2 lymph node dissection (dissection of the first and second lymph nodes) and surgery of curability A or B (as defined in the Japanese Classification of Gastric Carcinoma, 13th edition).
3) A case finally classified as stage II (excluding T1 cases), IIIA, or IIIB (as defined in the Japanese Classification of Gastric Carcinoma, 13th edition).
4) A case in which no hepatic, peritoneal, or distant metastasis was proven, and peritoneal cytodiagnosis was negative.
5) A case at the age of 20 to 80 (full) at enrollment.
6) A case which underwent no previous treatment (radiotherapy, chemotherapy, hormonal therapy, etc.) except surgical treatments.
7) A case which permits oral administration within 6 weeks after surgery.
8) A case which suffered no severe postoperative complication and had follow-up examination findings satisfying the following standards:

a. Leukocyte count: equal to or higher than the lower limit of each institution's standard or ≧4,000/mm³ (when the lower limit is in excess of 4,000/mm³);

b. Platelet count: ≧10×10⁴/mm³;

c. Total bilirubin: ≦1.5 mg/dL;

d. AST (GOT) and ALT (GPT): ≦2.5 times the upper limit of each institution's standard; and

e. Serum creatinine level: equal to or lower than the upper limit of each institution's standard.

9) A case which consented to written information about the subjection to the trial.

### (ii) Exclusion Criteria

Excluded are the following cases:

1) A case with synchronous or metachronous, double/multiple cancer. However, the following cases are eligible:

a. in the case of cervical carcinoma in situ or colorectal focal cancer in adenoma, even though the cancer is synchronous or metachronous double cancer;

b. in the case where the stage of the main lesion is not affected by complication lesions, even though the cancer is synchronous multiple cancer; and

c. in the case where the anamnestic lesion satisfies the eligibility conditions according to “Gastric cancer treatment guidelines (edition of March 2001),” and endoscopic mucosal dissection was performed, even though the cancer is metachronous multiple cancer (anamnestic gastric cancer).

2) A case of administration contraindication of TS-1 (see package insert).
3) A case which need taking flucytosine, phenytoin, or warfarin potassium.
4) A case which previously experienced severe drug allergy of Grade 3 or higher.
5) A case with severe complications (paralytic ileus, bowel obstructions, interstitial pneumonitis or pulmonary fibrosis, uncontrollable diabetes mellitus, heart failure, renal failure, liver failure, etc.).
6) A case involving diarrhea (watery diarrhea).
7) A case which is a pregnancy or lactation, a woman who may be pregnant, or a woman who wants to get pregnant.
8) A case which is a man who wants to have children in future.
9) A case of HIV positive.
10) Other cases which are recognized as an inadequate patient by doctors with responsibility or participation in this trial.

### (3) Treatment Methods

### 1) Surgery-Only Group (Control Group)

The patients assigned to the surgery-only group receive no anticancer treatment until metastasis or relapse is proven, and a follow-up examination is performed for 5 years after surgery.

The overall size of the control group was 530, of which 519 were eligible cases.

### 2) Surgery+TS-1 Administration Group (Trial Group)

To the patients assigned to the trial group, TS-1 is administered from within 45 days after surgery for one year from the surgery. The initial dose is defined by the body surface area (shown in Table 1). The daily initial dose is divided into two doses for taking after breakfast and evening meal. TS-1 has two types, classified by amount of Tegafur: 20 mg/capsule and 25 mg/capsule. An appropriate capsule is selected and administered in accordance with the body surface area as shown in Table 1.

Principally, the administration schedule includes drug administration for 28 consecutive days, followed by a rest period of 14 days. This administration schedule is made into one course and is repeated for one year after surgery. In each course, the consecutive days of administration does not exceed 28 days. The rest period must include 7 days or more. After passage of one year from the surgery, the patients do not undergo any new course. The patients are not received anticancer treatment until metastasis or relapse is proven, and a follow-up examination is performed for 5 years after surgery.

In order to ensure safety of the patients, a clinical examination is periodically (once/2 weeks) carried out during the TS-1 administration period, according to the package insert.

The overall size of the trial group was 529, of which 515 were eligible cases.

TABLE 1

| Body surface | Initial dose | After | After |
|---|---|---|---|
| area | of TS-1 | breakfast | evening meal |
| <1.25 m² | 80 mg/day | 40 mg | 40 mg |
| | | (20 mg × 2 cap) | (20 mg × 2 cap) |
| ≧1.25 m² and | 100 mg/day | 50 mg | 50 mg |
| <1.5 m² | | (25 mg × 2 cap) | (25 mg × 2 cap) |
| ≧1.5 m² | 120 mg/day | 60 mg | 60 mg |
| | | (20 mg × 3 cap) | (20 mg × 3 cap) |

Other details of the comparative trial are described in “Japanese Journal of Cancer and Chemotherapy” vol. 33, Supplement 1110-116, 2006.

### (4) Results

### 1) Overall Survival

Overall survival is measured from the date of the surgery until the date of the patient's death.

All the enrolled are tested, and the event includes deaths of cancer and those by other diseases. The survival duration of a survival-unidentified case is considered to be terminated at the end point when the survival has been proven. Note that when a patient died by an exceptional cause such as an accident, the survival duration is considered to be terminated at the time of death.

Table 2 shows the three-year survival rate of the subjects. FIG. 1 shows the change in the ratio of the number of all surviving patients to that of all eligible patients (overall survival rate). FIGS. 2, 3, and 4 show similar analytical results of stage II, IIIA, and IIIB patients, respectively.

TABLE 2

| | Hazard ratio | | | |
|---|---|---|---|---|
| Target | [95% CI] | Log-rank test | 3-year survival rate | |
| All | 0.66 | P = 0.0015 | Trial | 81% (n = 515) |
| | [0.51-0.85] | | Control | 70% (n = 519) |
| Stage II | 0.59 | P = 0.042 | Trial | 91% (n = 232) |
| | [0.36-0.99] | | Control | 82% (n = 233) |
| Stage IIIA | 0.66 | P = 0.032 | Trial | 77% (n = 194) |
| | [0.45-0.97] | | Control | 62% (n = 203) |
| Stage IIIB | 0.73 | P = 0.192 | Trial | 64% (n = 89) |
| | [0.45-1.18] | | Control | 57% (n = 83) |

As is clear from Table 2, the 3-year survival rate of the control group and that of the trial group were found to be 70% and 81%, respectively, with a significance (P=0.0015 by the log-rank test). In the case of stage II, the 3-year survival rate of the control group and that of the trial group were found to be 82% and 91%, respectively. In the case of stage IIIA, the 3-year survival rate of the control group and that of the trial group were found to be 62% and 77%, respectively. In both cases, significance was confirmed through the log-rank test. Therefore, a significantly high overall survival rate was attained for gastric cancer patients in stage II or stage IIIA, indicating that the therapeutic method of the present invention is particularly effective.

Adverse events were evaluated by means of NCI-CTC Ver. 2.0 (translated into Japanese by JCOG). In trial groups, the most common grades 3 and 4 adverse event was anorexia (6%). Thus, the therapeutic method of the invention was found to have remarkably high safety.

### 2) Relapse-Free Survival Rate

FIG. 5 shows the analytical results of the ratio of the number of surviving patients without relapse to all patients (relapse-free survival rate). As is clear from FIG. 5, the therapeutic method of the present invention provides a significantly high three-year relapse-free survival rate (p<0.0001), indicating that the therapeutic method of the present invention is effective.

### 3) Analysis in Terms of Sex and Age

FIG. 6 shows the analytical results of eligible patients by sex and by age.

As is clear from FIG. 6, the therapeutic method of the present invention is particularly effective for male patients. Regarding age, the therapeutic method of the present invention is particularly effective for patients under 60 years old.

### 4) Analysis by the TNM Classification

In the results shown Table 2, the eligible patients are classified by the stages defined by the Japanese Gastric Cancer Association. Meanwhile, the extent of gastric cancer is also classified by the TNM classification (UICC 5th Edition, 1997, Stomach (ICD-O C16)). Thus, the eligible patients were classified according to the TNM classification, and the survival rate of each stage was evaluated. The results are shown in FIGS. 7 and 8.

As is clear from FIGS. 7 and 8, the results with the TNM classification were equivalent to those obtained with the stage classification by Japanese Gastric Cancer Association. Notably, the therapeutic method of the present invention was particularly effective for gastric cancer patients in stage II by the TNM classification.

### 5) Histologic Type Analysis

The efficacy of the therapeutic method of the invention was evaluated on the basis of the histological examination results (differentiation status) of a tumor resected by the surgery. The examination was performed for the eligible patients. As shown in FIG. 8, the therapeutic method is more effective for patients of an undifferentiated tumor.

As described hereinabove, the therapeutic method of the present invention using TS-1 attains excellent results in terms of overall survival and safety.

1. A method for treatment of gastric cancer, which method is a postoperative adjuvant chemotherapy for gastric cancer, comprising orally administering, to a patient with gastric cancer in stage II, IIIA, or IIIB in a classification of the stage of gastric cancer, a pharmaceutical composition containing Tegafur, Gimeracil, and Oteracil potassium at a molar ratio of 1:0.4:1, at a dose of 50 to 150 mg/day as Tegafur dose and according to an administration schedule including, from within 45 days after a surgical operation of gastric cancer, drug administration for 28 consecutive days, followed by a rest period of 7 to 14 consecutive days.

2. A method for treatment according to claim 1, wherein the pharmaceutical composition is administered for one year after the surgical operation.

3. A method for treatment according to claim 1 or 2, wherein the patient suffers from gastric cancer in stage II or IIIA in a classification of the stage of gastric cancer.

4. A method for treatment according to claim 1 or 2, wherein the patient suffers from gastric cancer in stage II in a classification of the stage of gastric cancer.

5. A method for treatment according to claim 4, wherein the patient suffers from gastric cancer in which the depth of invasion is not T1.

6. A method for treatment according to claim 1 or 2, wherein the patient suffers from gastric cancer in stage IIIA in a classification of the stage of gastric cancer.

7. A method for treatment according to claim 1 or 2, wherein the patient suffers from gastric cancer in stage IIIB in a classification of the stage of gastric cancer.

8. A method for treatment according to claim 1 or 2, wherein the pharmaceutical composition is administered to the patient at a dose of 80 to 120 mg/day as Tegafur dose.

9. A method for treatment according to claim 1 or 2, wherein the pharmaceutical composition is administered to the patient at a daily dose divided into two doses.

10-27. (canceled)
